(19) United States
(12) Patent Application Publication
(10) Pub. No.: US 2023/0047535 A1
(43) Pub. Date: Feb. 16, 2023

(54) INDUCTION OF NEUROGENESIS USING UMBILICAL CORD DERIVED MESENCHYMAL STEM CELLS AND DERIVATIVES THEREOF

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); Amit N. PATEL, Miami, FL (US); Famela RAMOS, Oceanside, CA (US); James VELTMEYER, Oceanside, CA (US)

(73) Assignee: Therapeutic Solutions International, Inc., Oceanside, CA (US)

(21) Appl. No.: 17/886,407

(22) Filed: Aug. 11, 2022

Related U.S. Application Data
(60) Provisional application No. 63/232,137, filed on Aug. 11, 2021.

(51) Int. Cl. A61K 35/51; C12N 5/0775; A61P 25/00; C12N 5/077

(52) U.S. Cl. CPC A61K 35/51; C12N 5/0665; A61P 25/00; C12N 5/0653; C12N 5/0655; C12N 5/0654; C12N 5/0657; C12N 2501/24; C12N 2506/1369

(57) ABSTRACT

Disclosed are means, compositions of matter and protocols useful for treatment of neurological dysfunctions through stimulation of adult neurogenesis using administration of umbilical cord derived mesenchymal stem cells such as JadiCells. In one embodiment viral induced neuropathy is reduced by administration of JadiCells to stimulate neurogenesis. In another embodiment the neurogenic activity of selective serotonin reuptake inhibitors is enhanced by administration of JadiCells. In some embodiments administration of JadiCell exosomes, conditioned media, microvesicles and/or apoptotic bodies is utilized to stimulate neurogenesis.

### CROSS-REFERENCE TO RELATED APPLICATIONS

This application claims the benefit of priority to U.S. Provisional Application Ser. No. 63/232,137, filed on Aug. 11, 2021, the contents of which are incorporated herein by reference.

### FIELD OF THE INVENTION

The invention is directed to novel methods and compositions for utilizing regenerative cells for stimulating neurogenesis.

### BACKGROUND OF THE INVENTION

It is known that the central nervous system (CNS) is particularly vulnerable to insults that result in cell death or damage in part because cells of the CNS have a limited capacity for repair. Since damaged brain tissue does not regenerate, recovery must come from the remaining intact brain. Poor recovery from acute insults or chronic degenerative disorders in the CNS has been attributed to lack of neurogenesis, limited regeneration of injured nerves, and extreme vulnerability to degenerative conditions. The absence of neurogenesis was explained by the assumption that soon after birth the CNS reaches a permanently stable state, needed to maintain the equilibrium of the brain's complex tissue network. Research during the last decade showed, however, that the brain is capable of neurogenesis throughout life, albeit to a limited extent. In the inflamed brain, neurogenesis is blocked. This latter finding strengthened the traditional view that local immune cells in the CNS have an adverse effect on neurogenesis. Likewise, the limited regeneration and excessive vulnerability of CNS neurons under inflammatory conditions or after an acute insult were put down to the poor ability of the CNS to tolerate the immune-derived defensive activity that is often associated with local inflammation and cytotoxicity mediated, for example, by tumor necrosis factor (TNF)-.alpha. or nitric oxide. More recent studies have shown, however, that although an uncontrolled local immune response indeed impairs neuronal survival and blocks repair processes, a local immune response that is properly controlled can support survival and promote recovery. It was further shown that after an injury to the CNS, a local immune response that is well controlled in time, space, and intensity by peripheral adaptive immune processes (in which CD4.sup.+helper T cells are directed against self-antigens residing at the site of the lesion) is a critical requirement for post-traumatic neuronal survival and repair,

We know that neurogenesis occurs throughout life in adult individuals, albeit to a limited extent. Most of the newly formed cells die within the first 2-3 weeks after proliferation and only a few survive as mature neurons. Little is known about the mechanism(s) underlying the existence of neural stem/progenitor cells (NPCs) in an adult brain and why these cells are restricted in amount and limited to certain areas. Moreover, very little is known about how neurogenesis from an endogenous NPC pool can be physiologically increased. Knowledge of the factors allowing such stem cells to exist, proliferate, and differentiate in the adult individual is a prerequisite for understanding and promoting the conditions conducive to CNS repair. This in turn can be expected to lead to the development of interventions aimed at boosting neural cell renewal from the endogenous stem-cell pool or from exogenously applied stem cells.

### SUMMARY

Preferred embodiments include methods of stimulating neurogenesis in an adult comprising administration of a population of umbilical cord derived mesenchymal stem cells.

Preferred methods include embodiments wherein said neurogenesis occurs despite ongoing inflammation.

Preferred methods include embodiments wherein said ongoing inflammation is associated with increased levels of TNF-alpha as compared to an age-matched control.

Preferred methods include embodiments wherein said ongoing inflammation is associated with increased levels of IL-1 as compared to an age-matched control.

Preferred methods include embodiments wherein said ongoing inflammation is associated with increased levels of IL-6 as compared to an age-matched control.

Preferred methods include embodiments wherein said inflammation is associated with stimulation of a toll like receptor (TLR).

Preferred methods include embodiments wherein said TLR is TLR-1.

Preferred methods include embodiments wherein said TLR is TLR-3.

Preferred methods include embodiments wherein said TLR is TLR-2.

Preferred methods include embodiments wherein said TLR is TLR-4.

Preferred methods include embodiments wherein said TLR is TLR-5.

Preferred methods include embodiments wherein said TLR is TLR-7/8.

Preferred methods include embodiments wherein said TLR is TLR-9.

Preferred methods include embodiments wherein said neurogenesis is occurring in the dentate gyrus.

Preferred methods include embodiments wherein said neurogenesis is occurring in the subventricular zone.

Preferred methods include embodiments wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

Preferred methods include embodiments wherein the isolated cell expresses CD29, CD73, CD90, CD166, SSEA4, CD9, CD44, CD146, and CD105.

Preferred methods include embodiments wherein the isolated cell does not express CD45, CD34, CD14, CD79, CD106, CD86, CD80, CD19, CD117, Stro-1, and HLA-DR.

Preferred methods include embodiments wherein the isolated cell is positive for SOX2.

Preferred methods include embodiments wherein the isolated cell is positive for OCT4.

Preferred methods include embodiments wherein the isolated cell is positive for SOX2 and OCT4.

Preferred methods include embodiments wherein the wherein the isolated cell is capable of differentiation into a cell type selected from the group consisting of adipocytes, chondrocytes, osteocytes, cardiomyocytes, endothelial cells, and myocytes.

Preferred methods include embodiments wherein the isolated cell produces exosomes expressing CD63, CD9, or CD63 and CD9.

Preferred methods include embodiments wherein culturing comprises culturing in a culture media that is free of animal components.

Further embodiments are directed to cultures of differentiated cells derived from isolated cell, wherein the culture of differentiated cells includes a cell type selected from the group consisting of adipocytes, chondrocytes, osteocytes, cardiomyocytes, endothelial cells, myocytes and combinations thereof.

Preferred cultures include embodiments wherein the isolated cell has been differentiated into an adipocyte cell.

Preferred cultures include embodiments wherein the isolated cell has been differentiated into a chondrocyte cell.

Preferred cultures include embodiments wherein the isolated cell has been differentiated into an osteocyte cell.

Preferred cultures include embodiments wherein the isolated cell has been differentiated into a cardiomyocyte cell.

Preferred cultures include embodiments wherein the isolated cell has been expanded into a cell culture.

### BRIEF DESCRIPTION OF THE DRAWINGS

FIG. 1 is a bar graph showing varying levels of BrdU incorporation in mice after receiving either: a) bone marrow derived MSCs, b) adipose derived MSCs, or c) JadiCells

### DETAILED DESCRIPTION OF THE INVENTION

The invention provides the use of various stem cells for treatment of conditions lacking neurogenesis. In one embodiment, the invention teaches n one aspect, an isolated cell obtained from a subepithelial layer of a mammalian umbilical cord tissue capable of self-renewal and culture expansion is provided. Such a cell is capable of differentiation into a cell type such as, in one aspect for example, adipocytes, chondrocytes, osteocytes, cardiomyocytes, and the like. In another aspect, non-limiting examples of such cell types can include white, brown, or beige adipocytes, chondrocytes, osteocytes, cardiomyocytes, endothelial cells, myocytes, and the like, including combinations thereof. Other examples of such cell types can include neural progenitor cells, hepatocytes, islet cells, renal progenitor cells, and the like. A cross section of a human umbilical cord shows the umbilical artery (UA), the umbilical veins (UV), the Wharton's Jelly (WJ), and the subepithelial layer (SL). Isolated cells from the SL can have a variety of characteristic markers that distinguish them from cell previously isolated from umbilical cord samples. It should be noted that these isolated cells are not derived from the Wharton's Jelly, but rather from the SL.

Various cellular markers that are either present or absent can be utilized in the identification of these SL-derived cells, and as such, can be used to show the novelty of the isolated cells. For example, in one aspect, the isolated cell expresses at least three cell markers selected from CD29, CD73, CD90, CD146, CD166, SSEA4, CD9, CD44, CD146, or CD105, and the isolated cell does not express at least three cell markers selected from CD45, CD34, CD14, CD79, CD106, CD86, CD80, CD19, CD117, Stro-1, or HLA-DR. In another aspect, the isolated cell expresses at least five cell markers selected from CD29, CD73, CD90, CD146, CD166, SSEA4, CD9, CD44, CD146, or CD105. In another aspect, the isolated cell expresses at least eight cell markers selected from CD29, CD73, CD90, CD146, CD166, SSEA4, CD9, CD44, CD146, or CD105. In a yet another aspect, the isolated cell expresses at least CD29, CD73, CD90, CD166, SSEA4, CD9, CD44, CD146, and CD105. In another aspect, the isolated cell does not express at least five cell markers selected from CD45, CD34, CD14, CD79, CD106, CD86, CD80, CD19, CD117, Stro-1, or HLA-DR. In another aspect, the isolated cell does not express at least eight cell markers selected from CD45, CD34, CD14, CD79, CD106, CD86, CD80, CD19, CD117, Stro-1, or HLA-DR. In yet another aspect, the isolated cell does not express at least CD45, CD34, CD14, CD79, CD106, CD86, CD80, CD19, CD117, Stro-1, and HLA-DR. Additionally, in some aspects, the isolated cell can be positive for SOX2, OCT4, or both SOX2 and OCT4. In a further aspect, the isolated cell can produce exosomes expressing CD63, CD9, or both CD63 and CD9. A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate.

A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material.

The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1.times. glutamine, 1.times. NEAA, and 0.1-20% PRP lysate or platelet lysate. Another example of suitable media can include a base medium of DMEM low glucose without phenol red, 1.times. glutamine, 1.times. NEAA, 1000 units of heparin and 20% PRP lysate or platelet lysate. In another example, cells can be cultured directly onto a semi-solid substrate of DMEM low glucose without phenol red, 1.times. glutamine, 1.times. NEAA, and 20% PRP lysate or platelet lysate. In a further example, culture media can include a low glucose medium (500-1000 mg/mL) containing 1.times. Glutamine, 1.times. NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 1.times. Glutamine, 1.times. NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

In other embodiments, JadiCells are administered concurrently with adjuvants that may stimulation neurogeneic activity. Said Adjuvants include hCG, SSRISs and oxytocin.

### EXAMPLES

### JadiCell Administration is Superior to Other Stem Cells for Stimulating Dentate Gyrus Neurogenesis

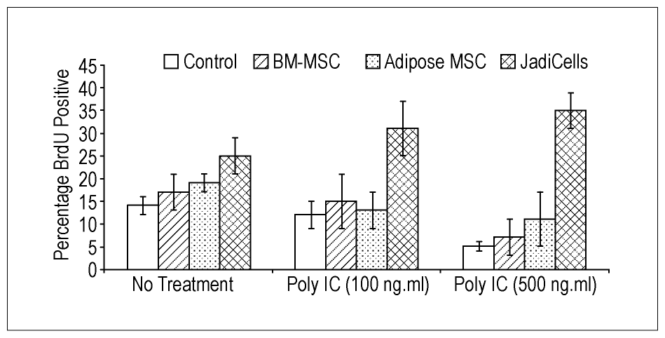

BALB/c mice were administered with 1 million of either bone marrow or adipose MSC or with JadiCells Intravenously at the same time as intraperitoneal injection of poly IC at the indicated concentrations. After a period of 3 days animals were given BrdU and sacrificed the next day. Proliferation was assessed based on BrdU incorporation. 10 animals per group were used. Results are shown in FIG. 1.

1. A method of stimulating neurogenesis in an adult comprising administration of a population of umbilical cord derived mesenchymal stem cells.

2. The method of claim 1, wherein said neurogenesis is occurring in the dentate gyrus.

3. The method of claim 1, wherein said neurogenesis is occurring in the subventricular zone.

4. The method of claim 1, wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

5. The isolated cell of claim 5, wherein the isolated cell expresses CD29, CD73, CD90, CD166, SSEA4, CD9, CD44, CD146, and CD105.

6. The isolated cell of claim 5, wherein the isolated cell does not express CD45, CD34, CD14, CD79, CD106, CD86, CD80, CD19, CD117, Stro-1, and HLA-DR.

7. The isolated cell of claim 5, wherein the isolated cell is positive for SOX2.

8. The isolated cell of claim 5, wherein the isolated cell is positive for OCT4.

9. The isolated cell of claim 5, wherein the isolated cell is positive for SOX2 and OCT4.

10. The isolated cell of claim 5, wherein the wherein the isolated cell is capable of differentiation into a cell type selected from the group consisting of adipocytes, chondrocytes, osteocytes, cardiomyocytes, endothelial cells, and myocytes.

11. The isolated cell of claim 5, wherein the isolated cell produces exosomes expressing CD63, CD9, or CD63 and CD9.

12. The isolated cell of claim 5, wherein culturing comprises culturing in a culture media that is free of animal components.

13. A culture of differentiated cells derived from the isolated cell of claim 16, wherein the culture of differentiated cells includes a cell type selected from the group consisting of adipocytes, chondrocytes, osteocytes, cardiomyocytes, endothelial cells, myocytes and combinations thereof.

14. The isolated cell of claim 5, that has been differentiated into an adipocyte cell.

15. The isolated cell of claim 5, that has been differentiated into a chondrocyte cell.

16. The isolated cell of claim 5, that has been differentiated into an osteocyte cell.

17. The isolated cell of claim 5, that has been differentiated into a cardiomyocyte cell.

18. The isolated cell of claim 5, that has been expanded into a cell culture.

19. The isolated cell of claim 5, that has been pretreated with an inflammatory stimuli.

20. The isolated cell of claim 5, that has been pretreated with interferon gamma.
